(19) United States
(12) Patent Application Publication
(10) Pub. No.: US 2024/0391947 A1
(43) Pub. Date: Nov. 28, 2024

(54) PROCESS FOR PREPARING (15alpha,16alpha,17beta)-ESTRA-1,3,5(10)-TRIENE-3,15,16,17-TETROL (ESTETROL) AND ESTETROL MONOHYDRATE

(71) Applicant: INDUSTRIALE CHIMICA S.R.L., Milan (IT)

(72) Inventors: Roberto Lenna, S. Giorgio Su Legnano (IT); Andrea Fasana, Nesso (IT)

(21) Appl. No.: 18/693,905

(22) PCT Filed: Oct. 1, 2021
(86) PCT No.: PCT/EP2021/077139

§ 371 Date: Mar. 20, 2024

(51) Int. Cl. C07J 1/00

(52) U.S. Cl. CPC C07J 1/007

(57) ABSTRACT

The present invention relates to a process for preparing (15α,16α,17β)-estra-1,3,5(10)-triene-3,15,16,17-tetrol, also known as Estetrol, and Estetrol monohydrate, having the formulas shown below:

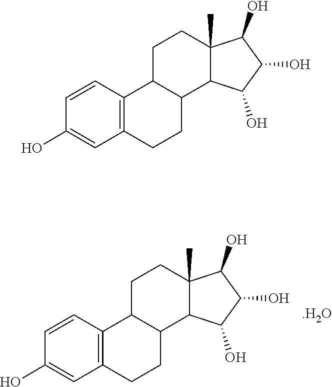 [SMILES: C[C@]12CCC3c4ccc(O)cc4CCC3C1[C@@H](O)[C@@H](O)[C@@H]2O ; C[C@]12CCC3c4ccc(O)cc4CCC3C1[C@@H](O)[C@@H](O)[C@@H]2O ; ".H2O"]

### FIELD OF THE INVENTION

The present invention refers to the sector of processes for the synthesis of active ingredients for pharmaceutical use, and in particular to a process for preparing the compound on an industrial scale (15α,16α,17β)-estra-1,3,5(10)-triene-3,15,16,17-tetrol, also known as Estetrol and in monohydrate form.

### BACKGROUND

The Estetrol compound is an active ingredient with pharmacological activity that makes it useful for Hormone Replacement Therapy (HRT), in female contraception, or in the therapy of autoimmune dysfunctions linked to hormonal imbalances.

The structural formula of Estetrol is reported below:

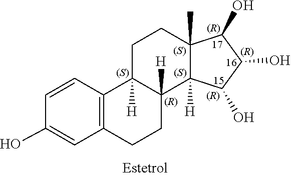

The positions 15, 16 and 17 of the steroidal skeleton (highlighted in the above reported formula) each bear one hydroxyl that, as indicated in the structural formula, have a defined spatial arrangement.

Estetrol is a natural product isolated from human urine and has been known for years; it has been described in the article “Synthesis of epimeric 15-hydroxyestriols, new and potential metabolites of estradiol”, J. Fishman et al., JOC Vol. 33, No. 8, August 1968, p. 3133-3135 (compound Ia of the figure on page 3133).

As far as the obtaining of Estetrol is concerned, the process obtainable from this article does not feature industrial applicability due to the low yield of the process.

Several patent applications have recently been published relating to new Estetrol synthesis processes but none of them avoids the formation of isomer 15β,16β,17β, having the structural formula shown below, from which Estetrol must be purified to be used in pharmaceutical preparations.

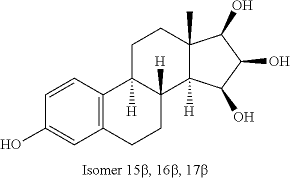

For example, application WO 2004/041839 A2 (page 6, lines 5-10) describes a process for obtaining Estetrol the purity of which can reach 99%, with the sum of the single impurities not exceeding 1%. Example 11 on page 28 describes an Estetrol with HPLC purity of 99.1% (HPLC-Ms) which however does not provide information on the content of the single impurities; the limit accepted by international guidelines for pharmaceutical substances is 0.1% for unknown ones and 0.15% for identified ones.

The content of impurities in an active ingredient (API) is an essential and non-derogable requirement to allow the use thereof in pharmaceutical preparations and is also a fundamental characteristic for defining an industrially applicable process. Any process, regardless of the yield, providing an API with an impurity content that does not respect the limits of the international guidelines is not an industrially useful process as the API, the result of the process, is not usable.

Subsequent applications relating to the production of Estetrol are, for example, WO 2012/164096 A1, WO 2013/050553 A1 and WO 2015/040051 A1.

In WO 2015/040051 A1 the ratio Estetrol/isomer 15β,16β,17β is equal to 99:1 in the examples 10 and 15, and equal to 98:2 in the examples 11 and 17. In these examples, however, no indication is given for lowering the content of isomer 15β,16β,17β to at least 0.15%. Even chromatographic purification (example 15) does not allow to obtain this result. In this document it is noted (page 9, lines 5-15) that the processes described in the discussed prior art (represented in the case of this document by applications WO 2012/164096 A1 and WO 2013/050553 A1) provide even higher and unacceptable amounts of isomer 15β,16β,17β.

It therefore appears clear that none of the described processes provides a solution to the limitation of the formation of the isomer 15β,16β,17β or a method of purification of Estetrol from said isomer.

### SUMMARY OF THE INVENTION

The object of the present invention is to provide an Estetrol and Estetrol monohydrate synthesis process with a content of isomer 15β,16β,17β lower than 0.15%, without having to resort to purification techniques that are not industrially applicable.

The invention relates to a synthesis process of Estetrol which comprises the following steps:
A) oxidation of compound (17β)-3-(phenylmethoxy)-estra-1,3,5(10),15-tetraen-17-ol (intermediate 1) to give compound (17β)-3-(phenylmethoxy)-estra-1,3,5(10)-triene-15,16,17-triol (intermediate 2):

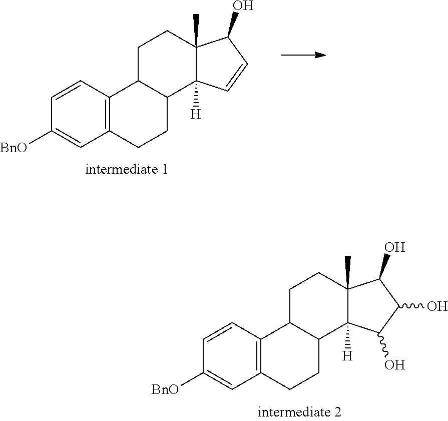

wherein Bn=benzyl, and in which the configuration of the carbon atoms 15 and 16 of the steroidal skeleton of intermediate 2 is not fixed;
B) debenzylation of the intermediate 2 to give compound (17β)-estra-1,3,5(10)-triene-3,15,16,17-tetrol (intermediate 3) in which the configuration of the carbon atoms 15 and 16 of the steroidal skeleton is not fixed:

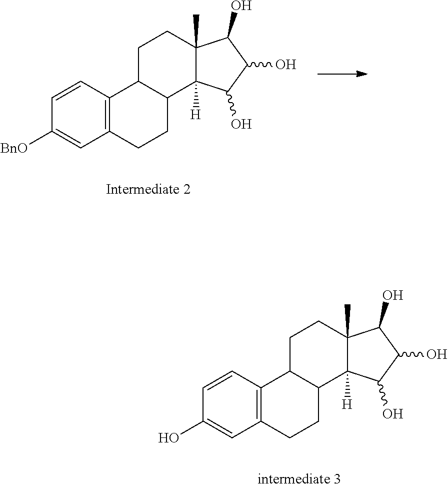 [SMILES: C[C@]12CCC3c4ccc(OCc5ccccc5)cc4CCC3[C@@H]1C(O)C(O)[C@@H]2O ; C[C@]12CCC3c4ccc(O)cc4CCC3[C@@H]1C(O)C(O)[C@@H]2O]

C) acetylation of intermediate 3 to (17β)-estra-1,3,5(10)-triene-3,15,16,17-tetrol tetraacetate (intermediate 4) in which the configuration of the carbon atoms 15 and 16 of the steroidal skeleton is not fixed:

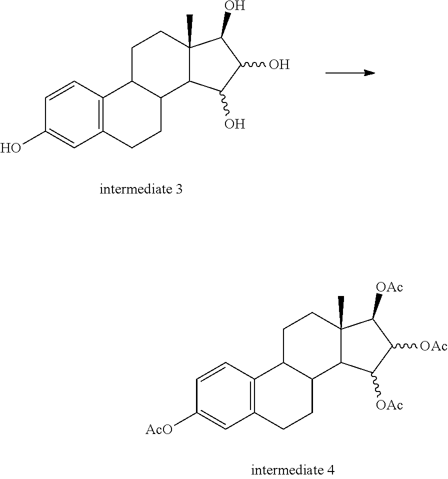 [SMILES: C[C@]12CCC3c4ccc(O)cc4CCC3C1C(O)C(O)[C@@H]2O ; CC(=O)Oc1ccc2c(c1)CCC1C2CC[C@@]2(C)C1C(OC(C)=O)C(OC(C)=O)[C@@H]2OC(C)=O]

D) purification of the intermediate 4 obtained in step C) to (15α,16α,17β)-estra-1,3,5(10)-triene-3,15,16,17-tetrol tetraacetate (intermediate 5) in which the configuration of the carbon atoms 15 and 16 of the steroidal skeleton is fixed:

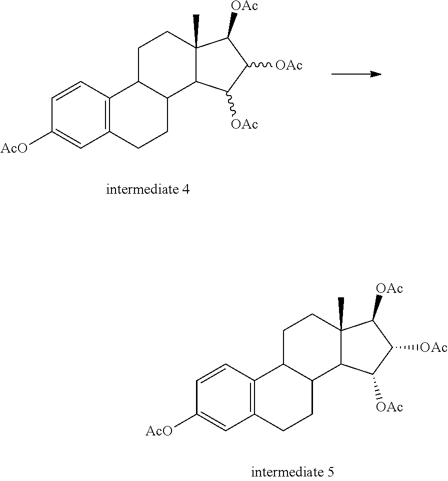 [SMILES: CC(=O)Oc1ccc2c(c1)CCC1C2CC[C@@]2(C)C1C(OC(C)=O)C(OC(C)=O)[C@@H]2OC(C)=O ; CC(=O)Oc1ccc2c(c1)CCC1C2CC[C@@]2(C)C1[C@@H](OC(C)=O)[C@@H](OC(C)=O)[C@@H]2OC(C)=O]

E) hydrolysis of the acetates present in the intermediate 5 to Estetrol:

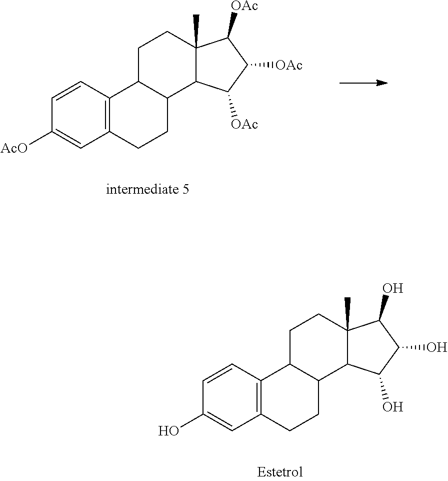 [SMILES: CC(=O)Oc1ccc2c(c1)CCC1C2CC[C@@]2(C)C1[C@@H](OC(C)=O)[C@@H](OC(C)=O)[C@@H]2OC(C)=O ; C[C@]12CCC3c4ccc(O)cc4CCC3C1[C@@H](O)[C@@H](O)[C@@H]2O]

F) reaction of Estetrol produced in step E) and transformation into Estetrol monohydrate:

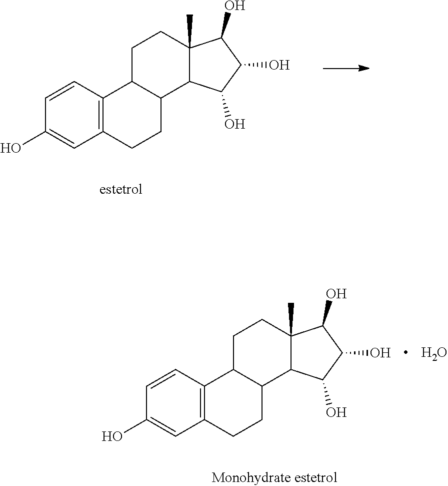 [SMILES: C[C@]12CCC3c4ccc(O)cc4CCC3C1[C@@H](O)[C@@H](O)[C@@H]2O ; C[C@]12CCC3c4ccc(O)cc4CCC3C1[C@@H](O)[C@@H](O)[C@@H]2O ; O]

### BRIEF DESCRIPTION OF THE DRAWINGS

FIG. 1 shows the HPLC chromatogram of Estetrol monohydrate obtainable with the process of the invention.

FIG. 2 shows the DRX diffractogram of Estetrol monohydrate obtainable with the process of the invention.

FIG. 3 shows the DSC curve of Estetrol monohydrate obtainable with the process of the invention.

### DETAILED DESCRIPTION OF THE INVENTION

The invention relates to a synthesis process of Estetrol and Estetrol monohydrate which comprises the steps defined above.

In the description that follows and in the claims when the term “reduced pressure” is used, it means a pressure lower than 0.5 bar; when the term “to small volume” is used with reference to an evaporation step, it is intended a residual volume of a solution less than 50% of the initial volume.

Step A) of the process of the invention consists in the oxidation of the compound (17β)-3-(phenylmethoxy)-estra-1,3,5(10),15-tetraen-17-ol (intermediate 1) to give the compound (17β)-3-(phenylmethoxy)-estra-1,3,5(10)-triene-15,16,17-triol (intermediate 2):

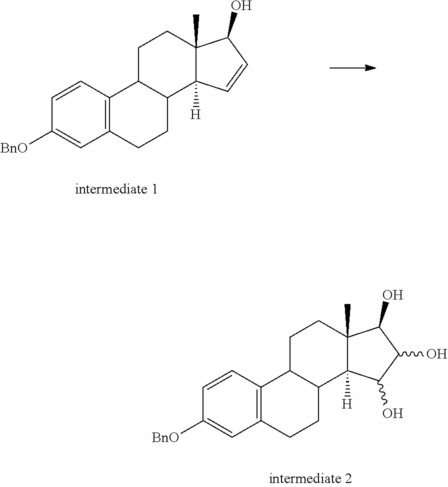

wherein Bn=benzyl, and in which the configuration of the carbon atoms 15 and 16 of the steroidal skeleton of intermediate 2 is not fixed.

The starting substrate of this step, intermediate 1, can be obtained as described in application WO 2004/041839 A2.

As oxidant in the reaction of step A) it is possible to use osmium tetroxide (OsO₄) supported on a polymer or, preferably, as such, or Potassium osmiate dihydrate K₂OsO₄·2H₂O. An organic amine N-oxide, such as trimethylamine N-oxide dihydrate, is used as co-oxidant.

Since oxidation with osmium derivatives is not stereoselective, intermediate 2 is obtained as a mixture of isomers with configuration 15α,16α,17β and 15β,16β,17β; the isomer 15α,16α,17β is produced in preponderant amount together with a minority amount of isomer 15β,16β,17β.

The reaction is carried out in a solvent inert to osmium derivatives, such as tetrahydrofuran (THF), at a temperature between 2° and 60° C., preferably between 30 and 50° C., and for a time of at least 12 hours, preferably at least 16 hours.

The reaction can be optionally made under inert atmosphere preferably under N₂

The reaction product (intermediate 2) after work up can be treated with a product sequestering metallic impurities in solution to eliminate the residual osmium content. These products, well known in chemistry, are generally based on a functionalized silica gel and commonly referred to in the sector by the term scavenger, which will be used in the rest of the text and the claims. The scavenger is preferably QuadraSil® MP.

The treatment with the scavenger can be carried out and can be repeated at each step of the process; it is preferably carried out in step F).

Step B) consists in the debenzylation of the intermediate 2 to give compound (17β)-estra-1,3,5(10)-triene-3,15,16,17-tetrol (intermediate 3) in which the configuration of the carbon atoms 15 and 16 of the steroidal skeleton is not fixed:

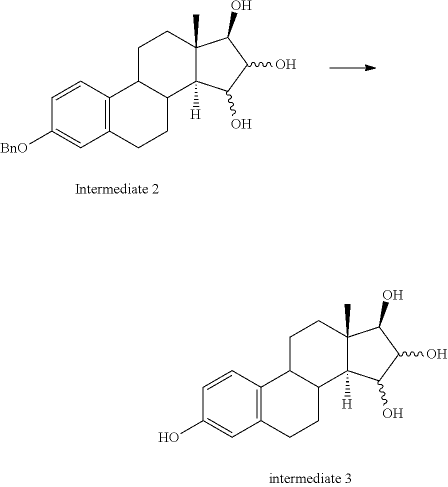 [SMILES: C[C@]12CCC3c4ccc(OCc5ccccc5)cc4CCC3[C@@H]1C(O)C(O)[C@@H]2O ; C[C@]12CCC3c4ccc(O)cc4CCC3[C@@H]1C(O)C(O)[C@@H]2O]

Debenzylation consists in hydrogenation with gaseous hydrogen in the presence of a suitable catalyst. Preferred conditions for this reaction are:
use of palladium on charcoal (Pd/C) at 5% or 10% by weight, preferably palladium on charcoal (Pd/C) at 5% by weight as a catalyst;
hydrogen pressure between 1 and 6 bar, preferably between 1 and 3 bar;
a linear or branched C1-C6 aliphatic alcohol, preferably methanol, as the reaction solvent;
reaction time of at least 12 hours, preferably at least 20 hours;
hydrogenation temperature between 10 and 60° C., preferably between 15 and 55° C., even more preferably between 20 and 50° C.

Step C) consists in acetylation of intermediate 3 to (17β)-estra-1,3,5(10)-triene-3,15,16,17-tetrol tetraacetate (intermediate 4) in which the configuration of the carbon atoms 15 and 16 of the steroidal skeleton is not fixed:

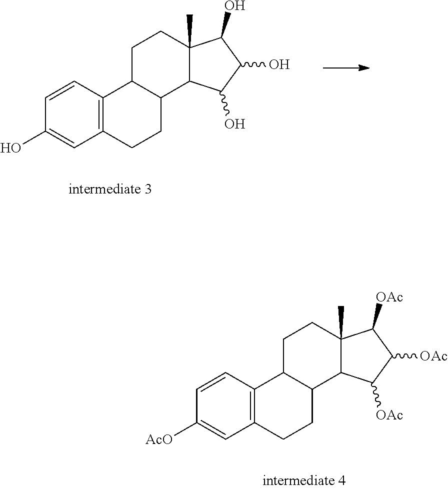 [SMILES: C[C@]12CCC3c4ccc(O)cc4CCC3C1C(O)C(O)[C@@H]2O ; CC(=O)Oc1ccc2c(c1)CCC1C2CC[C@@]2(C)C1C(OC(C)=O)C(OC(C)=O)[C@@H]2OC(C)=O]

The exhaustive acetylation of step C) is carried out in a solvent compatible with the conditions of the reaction, such as, for example, isopropyl acetate, ethyl acetate, tetrahydrofuran, pyridine or toluene. The preferred solvent is pyridine.

For the reaction acetic anhydride is used as reactant, in an amount of at least 4, preferably 6 moles per mole of intermediate 3, in the presence of an inorganic or organic base and of a catalyst.

Catalytic amounts of trifluoroacetic anhydride could be added.

Pyridine is preferably used as the organic base, and 4-dimethylaminopyridine (4-DMAP) as a catalyst.

The reaction temperature is between 5 and 40° C., preferably between 20 and 30° C.; the reaction time is at least 2 hours, preferably at least 3 hours.

The reaction can be optionally carried out under N₂ atmosphere.

Step D) consists in the purification of the intermediate 4 obtained in step C) to (15α,16α,17β)-estra-1,3,5(10)-triene-3,15,16,17-tetrol tetraacetate (intermediate 5) in which the configuration of the carbon atoms 15 and 16 of the steroidal skeleton is fixed:

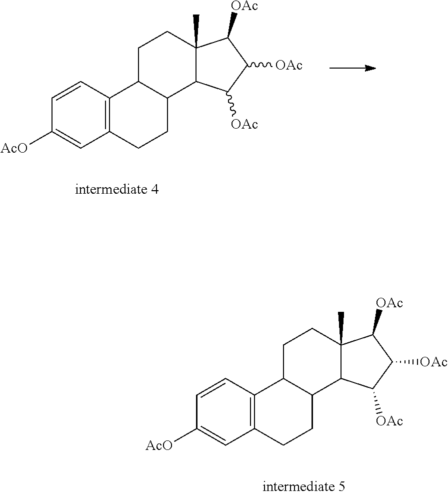 [SMILES: CC(=O)Oc1ccc2c(c1)CCC1C2CC[C@@]2(C)C1C(OC(C)=O)C(OC(C)=O)[C@@H]2OC(C)=O ; CC(=O)Oc1ccc2c(c1)CCC1C2CC[C@@]2(C)C1[C@@H](OC(C)=O)[C@@H](OC(C)=O)[C@@H]2OC(C)=O]

The purification of the intermediate 4, with elimination of the isomer 15β,16β,17β, is obtained with the sequence of operations described below:
D.1) dissolving intermediate 4 to be purified in DCM at 15-30° C.;
D.2) dripping the solution of intermediate 4 in DCM in pure methanol;
D.3) stirring the solution at 20-30° C. for at least 10 minutes;
D.4) distilling off the solvent under reduced pressure obtaining a suspension;
D.5) refluxing the suspension for at least 30′;
D.6) cooling to 20-25° C. and stirring for at least 1 h;
D.7) filtering intermediate 5 and drying at reduced pressure for at least 3 h at 40-60° C.

The purification treatment can be repeated the number of times necessary to obtain the desired level of purity according to the initial content of the isomer 15β,16β,17β.

The inventors carried out a series of experimental tests by repeating several times the sequence of operations D.1-D.7 on samples of intermediate 4 containing between 5 and 10% of isomer 15β,16β,17β getting a final product in which the content of isomer 15β,16β,17β was lower than 0.15% and in some cases lower than 0.05%.

Step E) consists in the hydrolysis of the acetates present in the intermediate 5 to Estetrol:

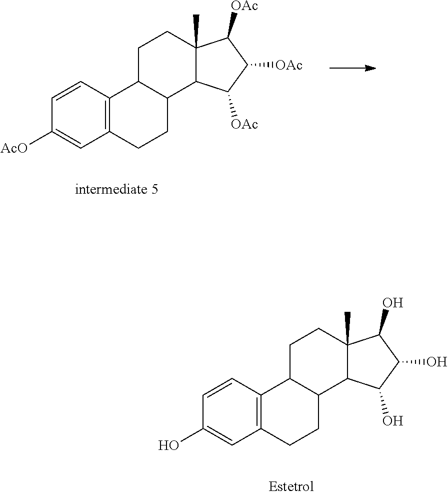 [SMILES: CC(=O)Oc1ccc2c(c1)CCC1C2CC[C@@]2(C)C1[C@@H](OC(C)=O)[C@@H](OC(C)=O)[C@@H]2OC(C)=O ; C[C@]12CCC3c4ccc(O)cc4CCC3C1[C@@H](O)[C@@H](O)[C@@H]2O]

The conditions of hydrolysis are those known to skilled person in organic chemistry.

The reaction of hydrolysis of the acetates of intermediate 4 has been made using bases in a solvent like a linear or branched C1-C6 aliphatic alcohol or a mixture thereof, preferably methanol. Preferred conditions for this reaction are:
use of sodium carbonate, potassium carbonate or lithium carbonate as a base; preferably potassium carbonate is used;
reaction time of at least 3 hours, preferably at least 4 hours;
reaction temperature between 10 and 40° C., preferably between 15 and 35° C., even more preferably between 20 and 30° C.

The solution containing the reaction product (Estetrol) can be optionally:
treated with a functionalized silica gel-based scavenger to eliminate the residual content of palladium; the scavenger is preferably QuadraSil® MP; and/or
purified through crystallization hot-cold in tetrahydrofuran (THF), methanol and acetonitrile, pure or as a mixture thereof.

In a second embodiment, the invention is directed to the preparation of Estetrol in monohydrate form. In this embodiment, the process comprises a further step, F),

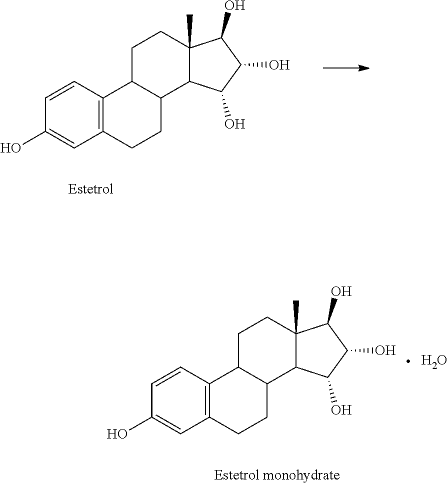 [SMILES: C[C@]12CCC3c4ccc(O)cc4CCC3C1[C@@H](O)[C@@H](O)[C@@H]2O ; C[C@]12CCC3c4ccc(O)cc4CCC3C1[C@@H](O)[C@@H](O)[C@@H]2O ; O]

which is carried out after step E) with the following sequence of operations:
F.1) dissolving Estetrol in a water-miscible organic solvent such as acetone, methanol, ethanol, isopropanol, tetrahydrofuran, dimethylformamide or dimethylacetamide until complete solution; the preferred solvent is methanol. In this operation, heating to reflux could be optionally performed for reaching a complete solution. The solution can be optionally treated with a functionalized silica gel-based scavenger to eliminate the residual content of palladium. The scavenger is preferably QuadraSil® MP. The solution can be optionally filtered on a Millipore membrane filter;
F.2) evaporating the solution obtained in operation F.1 under vacuum to small volume;
F.3) adding isopropyl alcohol (IPA), heating at 50-60° C. and evaporating under vacuum to small volume. Step F.3 (adding IPA and evaporating the solvent) can be repeated the number of times necessary to obtain the complete elimination of the solvent of step F.1;
F.4) adding isopropyl alcohol and heating to reflux (temperature higher than 75° C.) until complete solution has been obtained;
F.5) cooling the solution to 70-75° C.;
F.6) adding water (at least a volume equal to the volume of the organic solvent) and stirring at 60<T<70° C.;
F.7) distilling off the IPA under reduced pressure at 55<T<65° C.;
F.8) cooling the suspension to 0<T<5° C.;
F.9) stirring at 0<T<5° C. for at least 30 minutes;
F.10) filtering the solid and drying at 30<T<50° C. for at least 16 h under reduced pressure.

### Experimental Instruments, Methods and Conditions

### NMR:

NMR spectrometer JEOL 400 YH (400 MHz); JEOL Delta software v5.1.1;

Spectra recorded in DMSO-d₆.

### MS:

Instrument: DSQ-trace Thermofisher

Sample introduction—direct exposure probe (dep)

Chemical ionization (CI) with methane

Methane pressure: 2.2 psi

Source temperature: 200° C.

### HPLC:

Agilent Model 1260 Infinity chromatography system; UV Detector MODEL G1315C

DAD VL+

Method HPLC 1:
Chromatographic conditions:

| Column: | Supelco ascentis express C18 250 × 4.6 mm, 5 μm | |
|---|---|---|
| Flow: | 1 ml/min | |
| Detector: | UV 280 nm | |
| Injection volume: | 5 μl | |
| Temperature: | 25° C. | |
| Mobile phase A: | water | |
| Mobile phase B: | acetonitrile | |
| TIME | MOBILE PHASE | MOBILE PHASE |
| (min) | A (v/v) | B (v/v) |
| 0 | 80 | 20 |
| 0-5 | 80 | 20 |
| 5-45 | 20 | 80 |
| 45-55 | 20 | 80 |
| 55-56 | 80 | 20 |
| 56-66 | 80 | 20 |

Method HPLC 2:
Chromatographic conditions:

| | |
|---|---|
| Column: | Supelco discovery C18 150 × 4.6 mm, 5 μm |
| Flow: | 1 ml/min |
| Detector: | UV 280 nm |
| Injection volume: | 25 μl |
| Temperature: | 22° C. |
| Mobile phase A: | 4.29 g/L solution of CH₃COONH₄ in |
| | water/methanol/acetonitrile 90/6/4 |
| Mobile phase B: | 38.6 g/L solution of CH₃COONH₄ in |
| | water/methanol/acetonitrile 10/54/36 |

| TIME | MOBILE PHASE | MOBILE PHASE |
|---|---|---|
| (min) | A (v/v) | B (v/v) |
| 0 | 70 | 30 |
| 0-5 | 70 | 30 |
| 5-15 | 10 | 90 |
| 15-30 | 10 | 90 |
| 30-31 | 70 | 30 |
| 31-40 | 70 | 30 |

### UPLC:

Waters Acquity UPLC; Detector: Acquity UPLC PDA e λ Detector

Method UPLC:
Chromatographic conditions:

| Column: | Acquity UPLC BEH C18 1.7 μm, 2.1 × 50 mm | |
|---|---|---|
| Flow: | 0.5 ml/min | |
| Detector: | UV 225 nm | |
| Injection volume: | 1 μl | |
| Temperature: | 35° C. | |
| Mobile phase A: | water + 0.01% formic acid | |
| Mobile phase B: | acetonitrile + 0.01% formic acid | |
| TIME | MOBILE PHASE | MOBILE PHASE |
| (min) | A (v/v) | B (v/v) |
| 0 | 70 | 30 |
| 4-10 | 10 | 90 |
| 10-11 | 10 | 90 |
| 11-11.5 | 70 | 30 |
| 11.5-12 | 70 | 30 |

### UPLC-MS System:

Waters Acquity UPLC with Acquity UPLC PDA Detector connected to a Waters Acquity UPLC QDa Detector (ESI)

### TLC:

MERCK: TLC silica gel 60 F₂₅₄ Aluminum sheets 20×20 cm, code 1.0554.0001.

### TLC Detector:

Cerium phosphomolybdate: 25 g of phosphomolybdic acid and 10 g of cerium (IV) sulfate are dissolved in 600 mL of H₂O. 60 mL of 98% H₂SO₄ are added and the resulting mixture is brought to 1 L with H₂O. The plate is impregnated with the solution and then heated until the products are detected.

### XPRD:

The XRPD analysis was performed using a Bruker D2 Phaser (2nd edition) powder diffractometer operating in Bragg-Brentano geometry, equipped with a rotating multisampler and linear SSD type detector (Lynxeye). The X-ray source is an X-ray tube with a copper anode operated at 30 KV and 10 mA. For the analysis the X radiation having a wavelength corresponding to the average Kα of copper (λ=1.54184 Å) is used. The Kβ radiation is filtered through a nickel filter.

“Zero background” silicon sample holders with a flat surface were used on which the sample was spread to form a thin layer. During the analysis the sample holder is rotated at a speed of 60 rpm.

Scanning is performed in the 4-40° 2θ range with 0.016° 2θ increments and an acquisition time of 1.0 s for each increment.

The diffractograms were processed using the Bruker DIFFRAC.EVA software.

### DSC:

The DSC analysis was conducted in an inert atmosphere (nitrogen) using a Perkin Elmer Diamond DSC differential scanning calorimeter. Samples were prepared by weighing the powder into 40 μL aluminum crucibles, which were then sealed prior to analysis. The analysis was carried out in the temperature range 25-300° C. using a heating rate of 10° C./min.

### Notes

The water used in the experimental descriptions is to be understood as pure water unless otherwise indicated.

The organic solvents used in the experimental descriptions are to be understood as of “technical” grade unless otherwise indicated.

The reagents and catalysts used in the experimental descriptions are to be understood as of commercial quality unless otherwise indicated.

The product QuadraSil® MP is available from Johnson Matthey.

### Example 1

This example refers to step A) of the process of the invention, from intermediate 1 to intermediate 2.

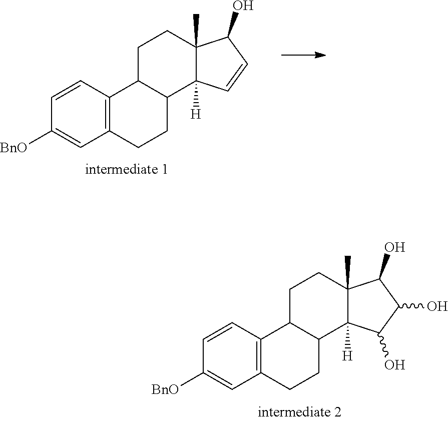 [SMILES: C[C@]12CCC3c4ccc(OCc5ccccc5)cc4CCC3[C@@H]1C=C[C@@H]2O ; C[C@]12CCC3c4ccc(OCc5ccccc5)cc4CCC3[C@@H]1C(O)C(O)[C@@H]2O]

In a flask under nitrogen, 32.4 g of intermediate 1 (89.87 mmol, 1 eq) and 356 mL of tetrahydrofuran were loaded. 0.324 g of osmium tetroxide (1.28 mmol, 1% by weight) and 17.9 g of trimethylamine N-oxide dihydrate (161.26 mmol, 1.8 eq) were added in this order to the solution. The system was heated to 50° C. and kept under stirring for 16 hours.

The reaction was controlled by TLC analysis under the following conditions: TLC plate: silica gel on alumina; starting substrate (intermediate 1) dissolved in dichloromethane; reaction mixture diluted in dichloromethane; eluent: ethyl acetate (EtOAc); detector: cerium phosphomolybdate.

At the end of the reaction, the solution was cooled to 25° C. and a solution of sodium metabisulphite (18.3 g) in water (162 mL) was dripped. The solvent was concentrated at reduced pressure and 193 mL of isopropyl acetate and 290 mL of 1M hydrochloric acid were added to the residue.

The phases were separated, and the aqueous phase was extracted with 160 mL of ethyl acetate. The organic solvent was washed with a solution of NaCl in water and the solution was dripped on 324 ml of pure n-heptane and stirred at 25° C. for 10 min (solution).

The ethyl acetate was removed under reduced pressure and the suspension obtained was stirred at 25° C. for 1 h.

The solid was filtered on büchner washing with n-heptane and dried at reduced pressure at 50° C. for 4 hours.

30 g of intermediate 2 was obtained and used as it is for the next step.

A small portion of intermediate 2 has been purified for analytical purpose, obtaining the following data:

¹H-NMR (400 MHz, DMSO-d₆): δ 7.31-7.43 (m, 5H); 7.15 (d, 1H, J=8.8 Hz); 6.72-6.75 (m, 1H); 6.69 (s, 1H); 5.04 (s, 2H); 4.86 (d, 1H, J=5.0 Hz); 4.61 (d, 1H, J=6.0 Hz); 4.27 (d, 1H, J=6.0 Hz); 3.67-3.73 (m, 2H); 3.25 (t, 1H); 2.74-2.77 (m, 2H); 1.03-2.22 (m, 9H); 0.67 (s, 3H).

Mass (ESI+): m/z=395 [M⁺+1], 377 [M⁺+1-H₂O].

### Example 2

This example refers to step B) of the process of the invention.

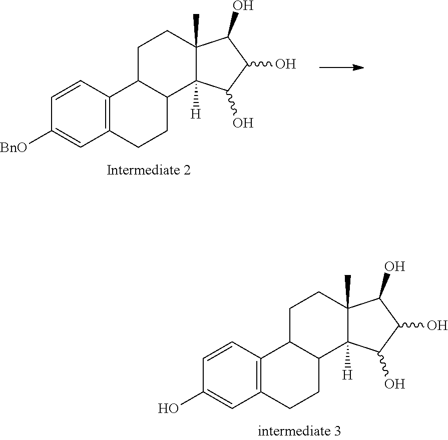 [SMILES: C[C@]12CCC3c4ccc(OCc5ccccc5)cc4CCC3[C@@H]1C(O)C(O)[C@@H]2O ; C[C@]12CCC3c4ccc(O)cc4CCC3[C@@H]1C(O)C(O)[C@@H]2O]

Intermediate 2 (8 g) obtained as described in the previous example was dissolved with 120 ml of methanol and loaded into a hydrogenation reactor. 800 mg of 5% palladium on charcoal were added to the solution and hydrogenation was carried out at 25° C. and 1 bar for 20 hours.

The reaction was controlled by TLC analysis under the following conditions: TLC plate: silica gel on alumina; starting substrate (intermediate 2) dissolved in dichloromethane (DCM); reaction mixture diluted with methanol (MeOH); eluent: DCM/MeOH 9/1; detector: cerium phosphomolybdate. At the end of the reaction, the system was filtered on a layer of dicalite washing with methanol.

The solvent was concentrated at reduced pressure to a residual volume of 20 mL and 60 ml of water were added (precipitation of solid has been detected).

The suspension was concentrated at reduced pressure to remove the residual methanol.

The suspension was stirred for 30 minutes at 20-25° C. The solid was filtered on büchner washing with water and dried at reduced pressure at 50° C. for 6 hours.

5.65 g of intermediate 3 (white solid) were obtained.

The amount of isomer 15β,16β,17β present in the reaction product has been determined by HPLC analysis and it is the 7.8% of the desired isomer 15α,16α,17β.

### Example 3

This example refers to the implementation of step C) of the process of the invention.

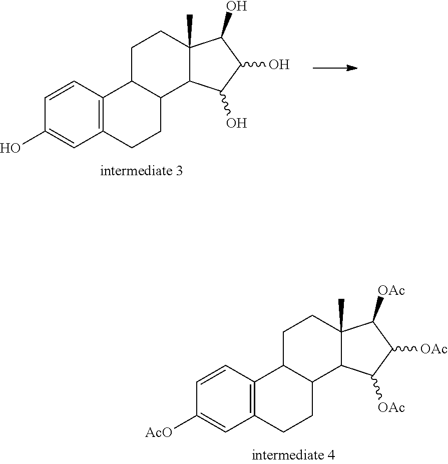 [SMILES: C[C@]12CCC3c4ccc(O)cc4CCC3C1C(O)C(O)[C@@H]2O ; CC(=O)Oc1ccc2c(c1)CCC1C2CC[C@@]2(C)C1C(OC(C)=O)C(OC(C)=O)[C@@H]2OC(C)=O]

5 g of intermediate 3 obtained as described in the previous example and 35 mL of pyridine were loaded into a flask and stirred at 20-25° C. (clear solution).

160 mg of 4-dimethylamino pyridine (4-DMAP) were added to the solution and, after 10′ of stirring at 20-25° C., 9.3 ml of acetic anhydride were added.

The reaction was controlled after 4 h of stirring at 25° C. by TLC analysis under the following conditions: TLC plate: silica gel on alumina; starting substrate (intermediate 3) dissolved in dichloromethane; reaction mixture diluted with HCl 1M and ethyl acetate (EtOAc); eluent: heptane/EtOAc 2/8; detector: cerium phosphomolybdate.

At the end of the reaction the solvent was concentrated at reduced pressure and 35 ml of DCM and 18 ml of water were added.

The biphasic system was kept under stirring at 25° C. for 10′ and then neutralized with 12M hydrochloric acid with cooling, and stirred for 30 minutes.

The organic solvent of the biphasic system was washed with a water solution of NaHCO₃ followed by a washing with water and, at the end, by a washing with a water solution of NaCl.

The solvent was distilled off completely at reduced pressure getting 7.4 g of raw intermediate 4 (solid).

The isomer 15β,16β,17β present in the raw intermediate 4 has been determined by HPLC (method 1) analysis and it is the 7.5% of the desired isomer 15α,16α,17β.

### Example 4

This example refers to the implementation of step D) of the process of the invention.

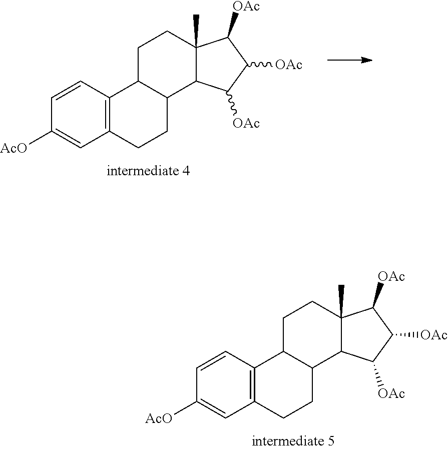 [SMILES: CC(=O)Oc1ccc2c(c1)CCC1C2CC[C@@]2(C)C1C(OC(C)=O)C(OC(C)=O)[C@@H]2OC(C)=O ; CC(=O)Oc1ccc2c(c1)CCC1C2CC[C@@]2(C)C1[C@@H](OC(C)=O)[C@@H](OC(C)=O)[C@@H]2OC(C)=O]

The raw intermediate 4 (7.4 g), obtained as described in the previous example, was dissolved in 29.6 mL of DCM (solution).

The DCM solution of intermediate 4 was dripped on 74 ml of pure methanol and stirred at 25° C. for 10′ (solution).

The organic solution was concentrated at 45° C. under reduced pressure to a final volume of 44 ml (suspension).

The suspension was refluxed (65° C.) for 30′ (suspension) then cooled at 25° C. under stirring for at least 1 h.

The solid was filtered on büchner washing with methanol, and dried at reduced pressure for 3 hours at 45° C.

The raw intermediate 4 (5.5 g; isomer 15β,16β,17β=0.17%) was dissolved in 22 mL of DCM (solution).

The DCM solution of intermediate 4 was dropped on 55 ml of pure methanol and stirred at 25° C. for 10′ (solution).

The organic solution was concentrated at 45° C. under reduced pressure to a final volume of 44 ml (suspension).

The suspension was refluxed (65° C.) for 30′ (suspension) then cooled at 25° C. under stirring for at least 1 h.

The solid was filtered on büchner washing with methanol, and dried at reduced pressure for 3 hours at 45° C.

5.2 g of pure intermediate 5 were obtained, which was analysed by HPLC.

The pure intermediate 5 had HPLC purity (method 1)=98.9%, with the isomer 15β,16β,17β not detectable.

¹H-NMR (400 MHz, CDCl₃): δ 7.27-7.26 (m, 1H); 6.83-7.86 (m, 1H); 6.79 (s, 1H); 5.39 (t, 1H); 5.16 (t, 1H); 5.01 (d, 1H, J=6.4 Hz); 2.83-2.87 (m, 2H); 2.28-2.23 (m, 2H); 2.28 (s, 3H); 2.09 (s, 3H); 2.05 (s, 6H); 1.50-1.85 (m, 7H); 0.94 (s, 3H).

Mass (ESI+): m/z=473 [M⁺+1], 413 [M⁺+1-AcOH], 353 [M⁺+1-2AcOH], 293 [M⁺+1-3AcOH].

### Example 5

This example refers to the implementation of step E) of the process of the invention.

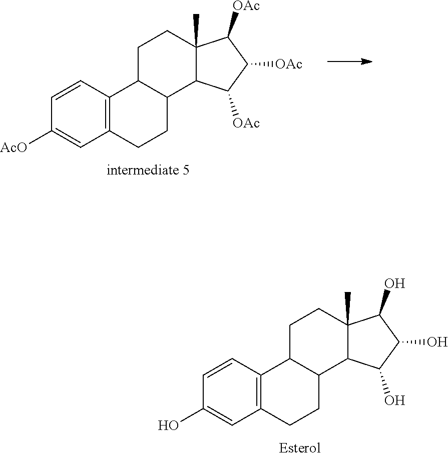 [SMILES: CC(=O)Oc1ccc2c(c1)CCC1C2CC[C@@]2(C)C1[C@@H](OC(C)=O)[C@@H](OC(C)=O)[C@@H]2OC(C)=O ; C[C@]12CCC3c4ccc(O)cc4CCC3C1[C@@H](O)[C@@H](O)[C@@H]2O]

2.5 g of intermediate 5 obtained in Example 4 were dissolved in 50 mL of methanol and 650 mg of potassium carbonate were added.

The mixture was kept under stirring at 25° C. for 4 hours (solution).

The reaction was controlled by TLC analysis under the following conditions: TLC plate: silica gel on alumina; intermediate product 5 dissolved in dichloromethane; reaction mixture quenched in 1M HCl and extracted with EtOAc, the organic phase was deposited; eluent: heptane/EtOAc 2/8; detector: cerium phosphomolybdate.

The solution was concentrated at reduced pressure to a residual volume of 5 mL, 18.5 mL of water were added, and the residual methanol was removed at reduced pressure.

The obtained suspension was neutralized with 1M hydrochloric acid (pH˜7) and cooled to 10° C. while stirring for 60 minutes. The solid was filtered on büchner washing with water and dried at reduced pressure at 50° C. for 6 hours.

1.5 g of Estetrol (white solid) were obtained.

Purity HPLC (method 2)=99.4%, isomer 15β,16β,17β not detectable.

Mass (ESI+): m/z=305 [M⁺+1], 287 [M⁺+1-H₂O], 269 [M⁺+1-2H₂O], 251 [M⁺+1-3H₂O].

### Example 6

This example refers to the implementation of step F) of the process of the invention.

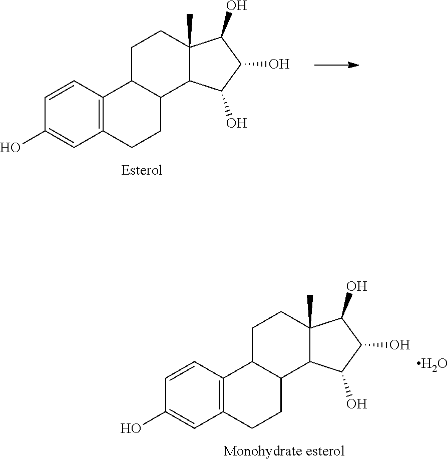

3.5 g of Estetrol obtained following the experimental procedures described in the previous examples were suspended under stirring in 63 mL of methanol.

The suspension was heated to reflux temperature to complete solution.

The solution was cooled at 20-25° C. and QuadraSil® MP was added keeping the solution under stirring for 16 h.

The slurry (solution of Estetrol—methanol and QuadraSil® MP) was filtered off.

The solution (Estetrol and methanol) was warmed to 45° C. and filtered on Millipore membrane filter, washing with MeOH.

The solution was evaporated at reduced pressure and 14 ml of Isopropyl alcohol (IPA) were added to the residual volume of 28 ml, keeping T≥50° C.

This last step was repeated two more times (final volume 28 ml).

Finally, 17.5 mL of IPA were added and the whole was refluxed until complete dissolution of the solids.

The solution was cooled to T=70° C. and 45.5 mL of water were added keeping T≥60° C. under stirring.

Slowly the suspension was distilled keeping T=55-65° C. under reduced pressure until residual volume of 35 mL.

The slurry was slowly cooled at 5° C., stirred for at least 30 minutes at this temperature and filtered on a büchner filter.

The filter cake was washed with water and the solid was dried in vacuum oven at 35° C. for about 18 h.

Estetrol monohydrate (white solid) was obtained (3.50 g; KF=5.72%)

The Estetrol monohydrate was analysed by HPLC (method 2).

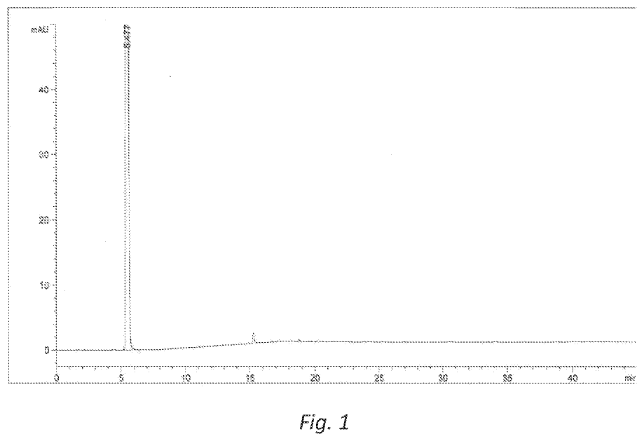

The results of the test are shown in FIG. 1: the product was found to be Estetrol monohydrate of HPLC purity=100%.

¹H-NMR (400 MHz, DMSO-d₆): δ 9.0 (s, 1H); 7.05 (d, 1H, J=8.4 Hz); 6.51-6.48 (m, 1H); 6.41 (d, 1H, J=2.4 Hz); 4.85 (d, 1H, J=4.8 Hz); 4.60 (d, 1H, J=5.6 Hz); 4.27 (d, 1H, J=6 Hz); 3.66-3.71 (m, 2H); 3.23-3.26 (t, 1H, J=5.6 Hz); 2.68-2.73 (m, 2H); 2.18-2.22 (m, 2H); 2.05-2.10 (m, 1H); 1.73-1.76 (d, 1H, 12 Hz); 1.02-1.45 (m, 5H); 0.66 (s, 3H).

Mass (ESI+): m/z=305 [M⁺+1], 287 [M⁺+1-H₂O], 269 [M⁺+1-2H₂O], 251 [M⁺+1-3H₂O].

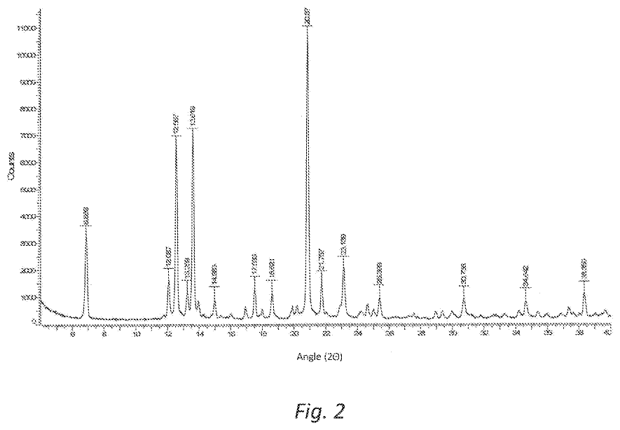

A sample of the product was subjected to XPRD analysis; the result of the test is the diffractogram shown in FIG. 2.

The list below reports the positions (as angle values 2θ±0.2°) and the relative intensities of the main peaks of the diffractogram:

6.889, 28.6%; 12.087, 13.2%; 12.567, 60.7%; 13.259, 8.7%; 13.619, 63.3%; 14.983, 6.6%; 17.533, 10.6%; 18.621, 9.1%; 20.871, 100.0%; 21.757, 11.8%; 23.139, 17.2%; 25.399, 7.2%; 30.736, 6.8%; 34.642, 6.0%; 38.359, 7.9%.

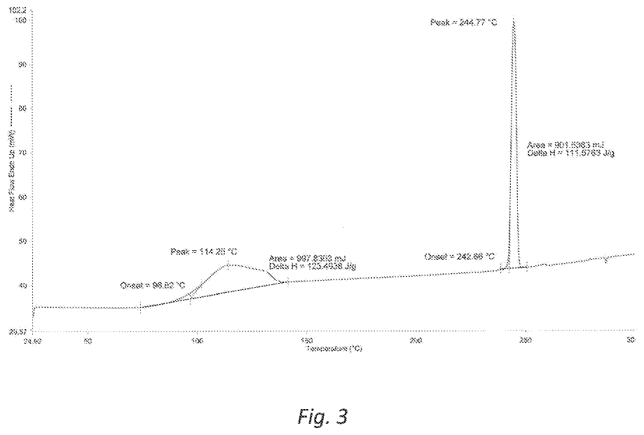

Another sample of the obtained product, weighing 3.4 mg, was subjected to DSC test; the result of the test is shown in FIG. 3, which shows a first widened peak attributed to the dehydration of Estetrol monohydrate, and a second peak at about 244-245° C., i.e. at a temperature essentially corresponding to the melting temperature of Estetrol.

What is claimed is:

1. A process for the synthesis of Estetrol and Estetrol monohydrate comprising the following steps:
A) oxidation of compound (17β)-3-(phenylmethoxy)-estra-1,3,5(10),15-tetraen-17-ol (intermediate 1) to give compound (17β)-3-(phenylmethoxy)-estra-1,3,5(10)-triene-15,16,17-triol (intermediate 2): 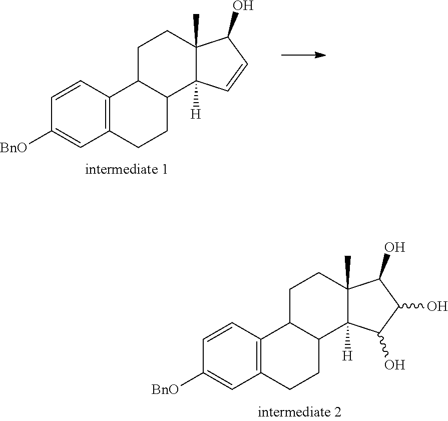
wherein Bn=benzyl, and in which the configuration of the carbon atoms 15 and 16 of the steroidal skeleton of intermediate 2 is not fixed;
B) debenzylation of the intermediate 2 to give compound (17β)-estra-1,3,5(10)-triene-3,15,16,17-tetrol (intermediate 3) in which the configuration of the carbon atoms 15 and 16 of the steroidal skeleton is not fixed: 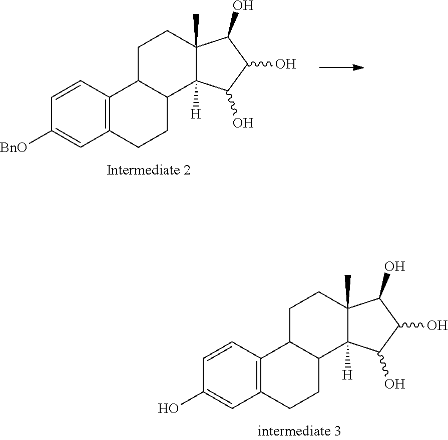 [SMILES: C[C@]12CCC3c4ccc(OCc5ccccc5)cc4CCC3[C@@H]1C(O)C(O)[C@@H]2O ; C[C@]12CCC3c4ccc(O)cc4CCC3[C@@H]1C(O)C(O)[C@@H]2O]
C) acetylation of intermediate 3 to (17β)-estra-1,3,5(10)-triene-3,15,16,17-tetrol tetraacetate (intermediate 4) in which the configuration of the carbon atoms 15 and 16 of the steroidal skeleton is not fixed: 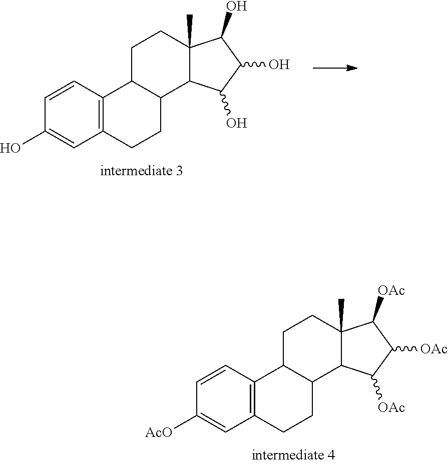 [SMILES: C[C@]12CCC3c4ccc(O)cc4CCC3C1C(O)C(O)[C@@H]2O ; CC(=O)Oc1ccc2c(c1)CCC1C2CC[C@@]2(C)C1C(OC(C)=O)C(OC(C)=O)[C@@H]2OC(C)=O]
D) purification of the intermediate 4 obtained in step C) to (15α,16α,17β)-estra-1,3,5(10)-triene-3,15,16,17-tetrol tetraacetate (intermediate 5) in which the configuration of the carbon atoms 15 and 16 of the steroidal skeleton is fixed: 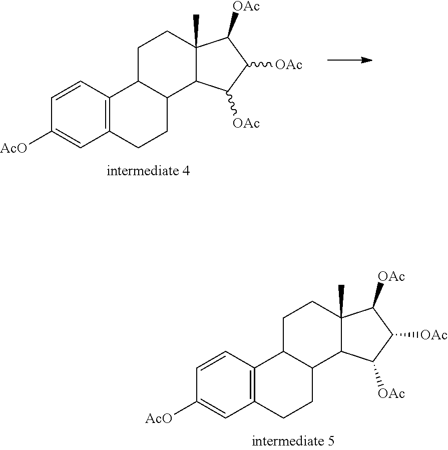 [SMILES: CC(=O)Oc1ccc2c(c1)CCC1C2CC[C@@]2(C)C1C(OC(C)=O)C(OC(C)=O)[C@@H]2OC(C)=O ; CC(=O)Oc1ccc2c(c1)CCC1C2CC[C@@]2(C)C1[C@@H](OC(C)=O)[C@@H](OC(C)=O)[C@@H]2OC(C)=O]
E) hydrolysis of the acetates present in the intermediate 5 to Estetrol 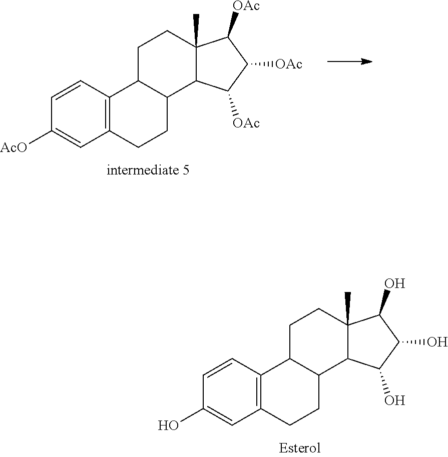 [SMILES: CC(=O)Oc1ccc2c(c1)CCC1C2CC[C@@]2(C)C1[C@@H](OC(C)=O)[C@@H](OC(C)=O)[C@@H]2OC(C)=O ; C[C@]12CCC3c4ccc(O)cc4CCC3C1[C@@H](O)[C@@H](O)[C@@H]2O]
F) transformation of Estetrol produced in step E) into Estetrol monohydrate

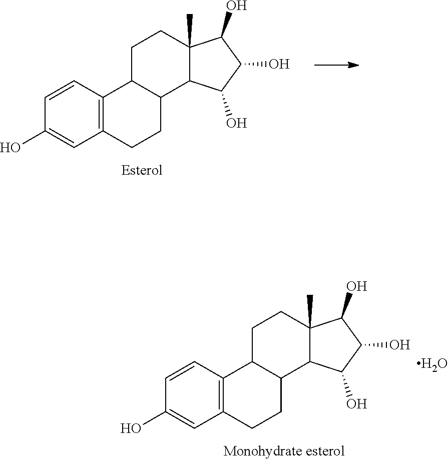

2. The process according to claim 1, wherein step A) is carried out using an osmium compound as such or supported on a polymer as an oxidant and an organic amine N-oxide as a co-oxidant, operating in a solvent inert to the derivatives of osmium, at a temperature between 20 and 60° C., and for a time of at least 12 hours.

3. The process according to claim 1, in which step B), the debenzylation reaction, is carried out under the following conditions:
use of palladium on charcoal (Pd/C) at 5% or 10% by weight as a catalyst;
hydrogen pressure between 1 and 3 bar;
a linear or branched C1-C6 aliphatic alcohol, as a reaction solvent;
reaction time of at least 12 hours;
hydrogenation temperature between 10 and 60° C.

4. The process according to claim 1, in which step C), the exhaustive acetylating reaction from intermediate 3 to intermediate 4, is carried out using acetic anhydride as a reactant in a solvent selected from isopropyl acetate, ethyl acetate, tetrahydrofuran, pyridine and toluene, in the presence of an inorganic or organic base, with trifluoroacetic anhydride or 4-dimethylaminopyridine (4-DMAP) as a catalyst and operating at a temperature between 5 and 40° C. for a time of at least 2 hours.

5. The process according to claim 1 in which step D), purification of intermediate 4 to give the intermediate 5, is carried out with the following sequence of operations:
D.1) dissolving the intermediate 4 to be purified in DCM at 15-30° C.;
D.2) dripping the solution of intermediate 4 in DCM in pure methanol;
D.3) stirring the solution of operation D.2) at 20-30° C. for at least 10 minutes;
D.4) distilling off the solvent under reduced pressure obtaining a suspension;
D.5) refluxing the suspension for at least 30′ (suspension);
D.6) cooling the suspension to 20-25° C. and stirring for at least 1 h;
D.7) filtering the intermediate 4 and drying at reduced pressure for at least 3 h at 40-60° C.

6. The process according to claim 1, wherein purification step D) is repeated the number of times necessary to obtain the desired level of purity according to the initial content of the isomer 15β,16β,17β.

7. The process according to claim 6, wherein said level of purity corresponds to a content of the isomer 15β,16β,17β≤0.15%.

8. The process according to claim 1, in which the hydrolysis reaction of step E), from intermediate 5 to Estetrol, is carried out under the following conditions:
use of sodium carbonate, potassium carbonate or lithium carbonate as a base in a solvent selected among linear or branched C1-C6 aliphatic alcohols or a mixture thereof;
reaction time of at least 3 hours;
reaction temperature between 10 and 40° C.

9. The process according to claim 1, in which in step F) Estetrol is transformed into Estetrol monohydrate with the following sequence of operations:
F.1) dissolving Estetrol in a water-miscible organic solvent;
F.2) evaporating under vacuum to the organic solvent to small volume;
F.3) adding isopropyl alcohol (IPA), heating at 50-60° C. and evaporating under vacuum to the organic solvent to small volume;
F.4) adding isopropyl alcohol and heating at reflux until complete solution has been obtained;
F.5) cooling the solution to 70-75° C.;
F.6) adding water and stirring at 60<T<70° C.;
F.7) distilling off the IPA under reduced pressure at 55<T<65° C. obtaining a suspension;
F.8) cooling the suspension to 0<T<5° C.;
F.9) stirring at 0<T<5° C. for at least 30 minutes;
F.10) filtering the solid and drying at 30<T<50° C. for at least 16 h under reduced pressure.

10. The process according to claim 1, in which the solution of operation F.1) is heated to reflux could until reaching a complete solution.

11. The process according to claim 10, wherein said solution is treated with a functionalized silica gel-based scavenger to eliminate the residual content of palladium.

12. The process according to claim 11, wherein said scavenger is QuadraSil® MP.
